(19) United States
(12) Patent Application Publication
(10) Pub. No.: US 2012/0177718 A1
(43) Pub. Date: Jul. 12, 2012

(54) WOUND-COVERING MATERIAL

(75) Inventors: Ryo Asato, Kyoto-shi (JP); Noriko Shinya, Kikuchi-shi (JP); Yuki Ishizawa, Kikuchi-shi (JP); Satomi Harano, Kikuchi-shi (JP)

(73) Assignee: THE CHEMO-SERO-THERAPEUTIC RESEARCH INSTITUTE, Kumamoto-shi (JP)

(21) Appl. No.: 13/377,757

(22) PCT Filed: Jun. 11, 2010
(86) PCT No.: PCT/JP10/59945

§ 371 Date: Dec. 12, 2011

(30) Foreign Application Priority Data
Jun. 11, 2009 (JP) 2009-140456

(51) Int. Cl. A61K 9/70; A61F 13/00; A61K 38/48

(52) U.S. Cl. 424/443; 424/94.64; 424/400; 602/48

(57) ABSTRACT

A new wound-covering material that protects and repairs a wound area caused by an operation, trauma, burn, and the like is provided. The present invention relates to a wound-covering material comprising thrombin, fibrinogen and a bioabsorbable supporting material. The wound-covering material may further be overlaid with a covering material for retaining moisture. The wound-covering material may be consisted of a kit of either (1) a bioabsorbable supporting material holding thrombin, and fibrinogen; or (2) a bioabsorbable supporting material holding thrombin, and a bioabsorbable supporting material holding fibrinogen; or (3) thrombin, fibrinogen and a bioabsorbable supporting material. Besides, each of the kits may be combined with a covering material for retaining moisture.

### TECHNICAL FIELD OF THE INVENTION

The present invention relates to a wound-covering material which comprises thrombin, fibrinogen and a bioabsorbable supporting material and which may easily be stuck to a deficient area in the skin caused by trauma, surgical operation, burn, and the like through adhering property of its own to thereby protect and repair a wound surface.

### BACKGROUND OF THE INVENTION

When deficiency occurs in the skin after surgical operation, trauma, and the like, material to cover and protect the wound surface is necessary. For a temporary measure, a gauze or an absorbent cotton may be used. However, a gauze or an absorbent cotton is problematic in that the wound surface may be destroyed when they are peeled off to cause bleeding again.

In recent years, various kinds of wound-covering material have been developed. For instance, there have been developed a film made of e.g. polyurethane and a wound-covering material called hydrocolloid dressing (e.g. Patent Reference 1). However, these are ones that do not positively support cure through auto-regeneration in the wound area but in principle is aimed mainly at maintaining moisture circumstances. Therefore, they basically depend on capacity of auto-regeneration and thus promotion of cure at the wound area would not be expected. Besides, due to their highly occlusive property, they may occasionally retain exudates in the suffered area to thereby retard cure.

In case of a full-thickness skin wound in the skin that is in a wide range and to the depth of the dermis, including a wound after surgery of removal of necrotic tissue for treatment of severe burn, cure of spontaneous regeneration would not be expected to necessitate transplantation of said tissue. For this purpose, autotransplantation or allotransplantation of the skin has hitherto been performed. However, in case of autotransplantation, of the skin, deficiency in the skin tissue is newly generated at the area where the skin was removed for transplantation to further necessitate regeneration of the skin at the deficient area. Besides, autotransplantation of the skin has restriction that it requires highly technical treatment in view of plastic surgery, that it is preferably applied to patients in good systemic conditions, and that it cannot be used in emergency. On the other hand, in case of allotransplantation of the skin, the skin is peeled off a week to 10 days after transplantation and thus is merely a temporary covering measure to necessitate transplantation of the skin from the patient.

In recent years, for regeneration of deficient skin, culture of epidermal cells alone to regenerate an epidermal layer (cultured epidermis) or transplantation of collagen sponge to the living body to regenerate dermis-like tissue (artificial dermis) has been performed (e.g. Non-patent References 1 and 2). However, the cultured epidermis may occasionally cause problems such as a low rate of take in case of severely deficient skin to the depth of the dermis as the cultured epidermis does not contain the dermis, and even in case of the take occurring, unevenness remains after closure of the wound, extreme promotion of wound contraction to cause scar contracture, suffering of pain, and the like. In case of the artificial dermis, there is also a problem of a low rate of take since animal-derived collagen is transplanted under the epidermis to thereby induce immune reaction to this substance and high infectivity. Also, some artificial dermis may require laborsome pretreatment such as repetition of immersion and washing in a saline before application.

In order to increase a rate of take, an approach has been developed where cultured dermis is spread over collagen gel or collagen sponge and thereupon epidermal cells are overlaid to construct a skin-like structure, which is transplanted (e.g. Patent Reference 2). However, this approach is also problematic in that, since living cells are used in an artificial skin with cultured cells, laborsome procedures are necessary for isolation of the cells, and transfer and storage of the cells are difficult. Furthermore, it will take time as long as several weeks for culture of the skin. Therefore, at the time when an artificial skin in an amount sufficient for covering the wound surface is obtained, the wound surface especially of burn has sometimes been afflicted with infection to render transplantation and the take of an artificial skin difficult.

Besides, the currently used wound-covering material and artificial skin basically lack adhesiveness and therefore sometimes may cause a problem when applied depending on a site or shape of a wound. When they are used with a supplementary fixing material such as an adhesive plaster and a film material, space for applying a fixing material becomes necessary to render their use at eyelids or lips inconvenient. When they are used with suture, the current dosage form of sponge with thickness would render their application to the wound surface in a complicated shape difficult. Skin trauma mostly occurs at fingers, limbs and face, all of which have a complicated, steric structure. Therefore, it is a great concern whether the currently used wound-covering material or artificial skin may protect the wound surface in compliance with the shape of the wound surface.
Patent reference 1: Japanese patent publication No. 9-262249
Patent reference 2: Japanese patent publication No. 6-292568
Non-patent reference 1: Ichiro Ono et al., Japan Surgical Society, 100(9): 522-529, 1999
Non-patent reference 2: Kazuya Matsuda et l., Burns, 17(2): 29-35, 1991

### DISCLOSURE OF THE INVENTION

### Technical Problem to be Solved by the Invention

For dissolving the problems mentioned above, one measure is to develop a wound-covering material that is capable of application as promptly as possible after a wound is produced, has adhesiveness of its own, is easily suited to a deficient area of the skin to be closely adhered thereto through elasticity and flexibility to protect the wound surface, and is capable of reconstructing deficient tissue of the skin in a prompt and good manner while relieving scar contracture and pain.

### Means for Solving the Problems

In view of the above-mentioned various problems, the present inventors have carried out intensive investigation and as a consequence found that a wound-covering material comprising thrombin and fibrinogen as an effective ingredient and a bioabsorbable supporting material as a substrate exerted extremely excellent effects to thereby complete the present invention.

Specifically, the present invention encompasses the following embodiments.

(1) A wound-covering material comprising thrombin and fibrinogen as an effective ingredient and a bioabsorbable supporting material as a substrate.
(2) The wound-covering material according to (1), wherein said wound-covering material is consisted of either

(i) a bioabsorbable supporting material holding thrombin, and fibrinogen; or

(ii) a bioabsorbable supporting material holding thrombin, and a bicabsorbable supporting material holding fibrinogen; or

(iii) thrombin, fibrinogen and a bioabsorbable supporting material;

wherein thrombin and fibrinogen are separated from each other immediately prior to use thereof.
(3) The wound-covering material according to (1) or (2), wherein said bioabsorbable supporting material is made of a material selected from the group consisting of polyglycolic acid, polylactic acid and a copolymer of glycolic acid and lactic acid.
(4) The wound-covering material according to any one of (1) to (3), wherein said bioabsorbable supporting material is processed to a non-woven fabric.
(5) The wound-covering material according to any one of (1) to (4), wherein said bioabsorbable supporting material is a non-woven fabric made of a material of polyglycolic acid.
(6) The wound-covering material according to any one of (1) to (5), wherein said wound-covering material comprises at least one additive selected from Blood Coagulation Factor XIII, a protease inhibitor, calcium chloride, albumin, sodium chloride, sodium citrate, or a non-ionic detergent.
(7) The wound-covering material according to any one of (1) to (6), wherein said wound-covering material optionally comprises a covering material for retaining moisture.
(8) The wound-covering material according to (7) wherein said covering material for retaining moisture is made of silicon or polyurethane.
(9) The wound-covering material according to any one of (1) to (8), wherein said wound-covering material may repair a deficient area in the skin due to trauma, surgical operation or burn.
(10) A wound-covering material kit comprising a bioabsorbable supporting material holding thrombin as an effective ingredient, and a container containing fibrinogen as an effective ingredient.
(11) The wound-covering material kit according to (10), wherein said bioabsorbable supporting material is made of a material selected from the group consisting of polyglycolic acid, polylactic acid and a copolymer of glycolic acid and lactic acid.
(12) The wound-covering material kit according to (10) or (11), wherein said bioabsorbable supporting material is processed to a non-woven fabric.
(13) The wound-covering material kit according to any one of (10) to (12), wherein said bioabsorbable supporting material is a non-woven fabric made of a material of polyglycolic acid.
(14) The wound-covering material kit according to any one of (10) to (13), wherein said wound-covering material comprises at least one additive selected from Blood Coagulation Factor XIII, a protease inhibitor, calcium chloride, albumin, sodium chloride, sodium citrate, or a non-ionic detergent.
(15) The wound-covering material kit according to any one of (10) to (14), wherein said calcium chloride is added to the bioabsorbable supporting material holding thrombin as an additive for thrombin.
(16) The wound-covering material kit according to any one of (10) to (14), wherein said Blood Coagulation Factor XIII is contained in the container containing fibrinogen.
(17) The wound-covering material kit according to any one of (10) to (16), wherein said bioabsorbable supporting material is prepared by a process comprising the step of immersing a bioabsorbable supporting material in a solution containing thrombin, and the step of lyophilizing the supporting material obtained by said step.
(18) The wound-covering material kit according to any one of (10) to (17), wherein said wound-covering material optionally comprises a covering material for retaining moisture.
(19) The wound-covering material kit according to (18) wherein said covering material for retaining moisture is made of silicon or polyurethane.
(20) The wound-covering material kit according to any one of (10) to (19), wherein said wound-covering material may repair a deficient area in the skin due to trauma, surgical operation or burn.
(21) A wound-covering material kit comprising a bioabsorbable supporting material holding thrombin as an effective ingredient, and a bioabsorbable supporting material holding fibrinogen as an effective ingredient.
(22) The wound-covering material kit according to (21), wherein said bioabsorbable supporting material is made of a material selected from the group consisting of polyglycolic acid, polylactic acid and a copolymer of glycolic acid and lactic acid.
(23) The wound-covering material kit according to (21) or (22), wherein said bioabsorbable supporting material is processed to a non-woven fabric.
(24) The wound-covering material kit according to any one of (21) to (23), wherein said bioabsorbable supporting material is a non-woven fabric made of a material of polyglycolic acid.
(25) The wound-covering material kit according to any one of (21) to (24), wherein said wound-covering material comprises at least one additive selected from Blood Coagulation Factor XIII, a protease inhibitor, calcium chloride, albumin, sodium chloride, sodium citrate, or a non-ionic detergent.
(26) The wound-covering material kit according to any one of (21) to (25), wherein said calcium chloride is added to the bioabsorbable supporting material holding thrombin as an additive for thrombin.
(27) The wound-covering material kit according to any one of (21) to (25), wherein said non-ionic detergent is contained in the bioabsorbable supporting material holding fibrinogen.
(28) The wound-covering material kit according to any one of (21) to (27), wherein said bioabsorbable supporting material holding thrombin or said bioabsorbable supporting material holding fibrinogen is prepared by a process comprising the step of immersing a bioabsorbable supporting material in a solution containing thrombin or fibrinogen, and the step of lyophilizing the supporting material obtained by said step.
(29) The wound-covering material kit according to any one of (21) to (28), wherein said wound-covering material optionally comprises a covering material for retaining moisture.
(30) The wound-covering material kit according to (29) wherein said covering material for retaining moisture is made of silicon or polyurethane.
(31) The wound-covering material kit according to any one of (21) to (30), wherein said wound-covering material may repair a deficient area in the skin due to trauma, surgical operation or burn.
(32) A wound-covering material kit comprising a container containing thrombin as an effective ingredient, a container containing fibrinogen as an effective ingredient, and a bioabsorbable supporting material.
(33) The wound-covering material kit according to (32), wherein said bioabsorbable supporting material is made of a material selected from the group consisting of polyglycolic acid, polylactic acid and a copolymer of glycolic acid and lactic acid.
(34) The wound-covering material kit according to (32) or (33), wherein said bioabsorbable supporting material is processed to a non-woven fabric.
(35) The wound-covering material kit according to any one of (32) to (34), wherein said bioabsorbable supporting material is a non-woven fabric made of a material of polyglycolic acid.
(36) The wound-covering material kit according to any one of (32) to (35), wherein said wound-covering material comprises at least one additive selected from Blood Coagulation Factor XIII, a protease inhibitor, calcium chloride, albumin, sodium chloride, sodium citrate, or a non-ionic detergent.
(37) The wound-covering material kit according to any one of (32) to (36), wherein said calcium chloride is added to the container containing thrombin as an additive for thrombin.
(38) The wound-covering material kit according to any one of (32) to (36), wherein said Blood Coagulation Factor XIII is contained in the container containing fibrinogen.
(39) The wound-covering material kit according to any one of (32) to (38), wherein said wound-covering material optionally comprises a covering material for retaining moisture.
(40) The wound-covering material kit according to (39) wherein said covering material for retaining moisture is made of silicon or polyurethane.
(41) The wound-covering material kit according to any one of (32) to (40), wherein said wound-covering material may repair a deficient area in the skin due to trauma, surgical operation or burn.

### EFFECTS OF THE INVENTION

It was revealed that a wound-covering material according to the present invention has the following properties and hence may be an ideal wound-covering material.

It may be applied as promptly as possible after a wound is produced since culture and pretreatment (repetition of immersion and washing with a saline) are riot necessary;

It may be applied through adhesiveness of its own due to fibrin formation and hence suture may be unnecessary;

With elasticity and flexibility, it may be closely attached to a wound surface in a complicated shape;

As a result of an adhering and sealing effect, storage of exudates may be inhibited and pain may be alleviated;

It may advantageously be used for fresh trauma accompanied by bleeding since it may inhibit bleeding through sealing effect;

Through the activity of thrombin to promote cell growth, it may promote reconstruction of deficient tissue including the epidermis together with formation of granulation tissue;

After application to a wound surface, it may secure space where granulation tissue is formed with an appropriate strength, may inhibit wound contraction and may decrease scar contracture;

In case that a covering material for retaining moisture is not overlaid, it may serve as a wound-covering material as protecting a wound surface and being naturally peeled off after cure; in case that a covering material for retaining moisture is optionally overlaid to provide moisture circumstances, it may serve as an artificial skin wherein the rest other than a covering material for retaining moisture serves as a scaffold for cell growth to be replaced with auto-tissue; and

It is excellent in safety.

### BRIEF DESCRIPTION OF DRAWINGS

FIG. 1 is a photograph of histopathology on Day 10 after application of the wound-covering material of the present invention.

FIG. 2 is a photograph of histopathology, after application of the wound-covering material of the present invention, on Week 3 after being brought to moisture circumstances by covering a covering material for retaining moisture.

### BEST MODE FOR CARRYING OUT THE INVENTION

The present invention relates to a wound-covering material comprising thrombin and fibrinogen as an effective ingredient and a bioabsorbable supporting material as a substrate. The wound-covering material may optionally comprise a covering material for retaining moisture.

The bioabsorbable supporting material for use in the present invention may be any bioabsorbable synthetic fiber. A bioabsorbable synthetic fiber as used herein refers to a synthetic fiber that is unlikely to induce inflammation in the living body as foreign substance and may be absorbed and/or degraded within the living body with time. The bioabsorbable supporting material has preferably appropriate flexibility and elasticity to ensure that it may surely cover any deficient area in any shape. The bioabsorbable supporting material may preferably be processed into a non-woven fabric. The non-woven fabric may be prepared e.g. by making bioabsorbable material into woven or knitted fabric and then needle-punched to give a non-woven fabric in accordance with the method described in Japanese Patent Publication No. 18579/1993. For example, a synthetic fiber that may form such a non-woven fabric includes polyglycolic acid, polylactic acid, or a copolymer of glycolic acid with lactic acid, etc., which may be used after processing into a non-woven fabric. Among these, a bioabsorbable synthetic non-woven fabric which is prepared from polyglycolic acid by processing into a non-woven fabric is the most preferable material for the purpose of the present invention.

The bioabsorbable supporting material may be in any shape but preferably in the form of a sheet in view of versatility to deficient area of the skin.

In addition to thrombin and fibrinogen as the effective ingredients, a pharmaceutically acceptable stabilizer and additive may also be added. Examples of such stabilizer and additive include, for instance, Blood Coagulation Factor XIII, a protease inhibitor, calcium chloride, albumin, sodium chloride, sodium citrate, a non-ionic detergent, mannitol, and the like.

Thrombin, fibrinogen and Blood Coagulation Factor XIII may preferably be derived from human blood or obtained by the genetic engineering. The wound-covering material of the present invention may be in any dosage form so far as thrombin and fibrinogen as an effective ingredient are ultimately contained in a bioabsorbable supporting material. In view of easy handling under operative settings, however, a bioabsorbable supporting material previously holding thrombin and/or fibrinogen, which maintains flexibility, is one of preferable embodiments from the viewpoint of its easy handling as well as tissue sealing efficacy.

In case that a bioabsorbable supporting material previously holds both thrombin and fibrinogen, the bioabsorbable supporting material should hold each of thrombin and fibrinogen under such condition that the components are separated from each other or each of the components in the form of powder are suspended in an organic solvent and each suspension is sprayed to the non-woven fabric, so that both thrombin and fibrinogen may not react to each other to generate stabilized fibrin.

The kit of the present invention may comprise either:

(A) a bioabsorbable supporting material holding thrombin plus fibrinogen; or
(B) a bioabsorbable supporting material holding thrombin and a bioabsorbable supporting material holding fibrinogen; or
(C) a bioabsorbable supporting material, thrombin, and fibrinogen;
in which a stabilizer and an additive as described above may optionally be added to either of (A) to (C). Namely, the kit of the present invention may comprise embodiments where a bioabsorbable supporting material holds thrombin alone (A), where a bioabsorbable supporting material holds both thrombin and fibrinogen (B), and where a bioabsorbable supporting material holds neither thrombin nor fibrinogen (C).

For use in case of (A), after fibrinogen is applied to a wound surface, a bioabsorbable supporting material holding thrombin (hereinafter referred to as “supporting material holding thrombin”) is overlaid, or alternatively, fibrinogen is applied to a supporting material holding thrombin by spraying or by immersing. Said supporting material holding thrombin may be prepared by (1) dissolving thrombin in a saline or a buffer and optionally adding to the resulting thrombin solution calcium chloride as an additive as appropriate, and (2) immersing the supporting material into said thrombin solution, followed by freezing at −80° C. for 2 hours and lyophilization. An amount of thrombin to be held on a supporting material holding thrombin may preferably be 20-100 U/cm².

For use in case of (B), a bioabsorbable supporting material holding fibrinogen (hereinafter referred to as “supporting material holding fibrinogen”) and a supporting material holding thrombin are overlaid to each other, and to the supporting material holding fibrinogen put upside is added dropwise distilled water, the surface of which supporting material holding fibrinogen is applied to an afflicted area. Said supporting material holding fibrinogen may be prepared by (1) dissolving fibrinogen in a saline or a buffer and optionally adding to the resulting fibrinogen solution a non-ionic detergent as an additive as appropriate, and (2) immersing the supporting material into said fibrinogen solution, followed by freezing at −80° C. for 2 hours and lyophilization. An amount of fibrinogen to be held on a supporting material holding fibrinogen may be 0.5-5 mg/cm², a range of concentration where fibrinogen may exert a hemostatic activity, and preferably 1-4 mg/cm².

For use in case of (C), after fibrinogen, prepared as in the process for preparing a commercially available fibrin sealant (e.g. Bolheal manufactured by Juridical Foundation The Chemo-Sero-Therapeutic Research Institute), is applied to a wound surface, a bioabsorbable supporting material immersed into the solution of thrombin is applied, or alternatively, each of the solutions of thrombin and of fibrinogen is applied simultaneously to the supporting material via spray.

In either case of (A) to (C), Blood Coagulation Factor XIII or a protease inhibitor may be added to a solution containing fibrinogen.

The wound-covering material of the present invention, when applied to a wound surface, may adhere to and protect the wound surface and may be naturally peeled off after cure. In case that an afflicted area is under moisture condition, the wound-covering material may serve as an artificial skin as serving as a scaffold for cell growth to be replaced with auto-tissue.

In case that a wound reaches to the depth of the skin or is spread in a wide range, or in case that an afflicted area is not under moisture condition, a covering material for retaining moisture may preferably be used which is overlaid to the wound-covering material as above. As a consequence, a wound surface may be kept under moisture condition so that the rest other than the covering material for retaining moisture may serve as a scaffold for cell growth to be replaced with auto-tissue. The covering material for retaining moisture as used herein may be any material as far as it may sufficiently retain moisture and may be non-toxic to cells and includes preferably silicon membrane, urethane membrane, and the like.

The wound-covering material obtained in accordance with the present invention, due to its high adhesiveness, appropriate strength, flexibility and elasticity, may be stuck to a deficient area of the skin in any shape to alleviate scar contracture and pain. Besides, since every material used therein is safe to the living body, it may be used in clinical without care.

The present invention is explained in more detail by means of the following Examples but should not be construed to be limited thereto.

### EXAMPLE

### Preparation Example 1

### Preparation of Supporting Material Holding Thrombin

Thrombin contained in a commercially available tissue sealant kit, Bolheal, Juridical Foundation The Chemo-Sero-Therapeutic Research Institute, was dissolved in a dissolving solution attached to the kit to prepare a thrombin solution at 1875 U/mL of thrombin. Using this thrombin solution at 1875 U/mL of thrombin, a thrombin solution (pH 6.0) was prepared containing 1% glycerol, 3% trehalose, 0.18 M histidine, 40 mM calcium chloride and 0.1% Tween 80, at a final concentration. The thrombin solution (2.5 mL) was soaked evenly into a bioabsorbable synthetic non-woven fabric made of polyglycolic acid (Neoveil, Gunze Limited, 5×10 cm, thickness 0.15 mm). This sample, after being frozen and lyophilized for 24 hours, was used as a sample of a supporting material holding thrombin (thrombin held at 93.8 U/cm²).

### Preparation Example 2

### Preparation of Supporting Material Holding Fibrinogen

Using fibrinogen contained in a commercially available tissue sealant kit, Bolheal, Juridical Foundation The Chemo-Sero-Therapeutic Research Institute, a 8% fibrinogen solution was prepared containing Tween 80 (0.1%) as a non-ionic detergent, albumin (1.0%), NaCl (1.75%), citric acid 3Na (1.2%), glycine (1.5%), and D-mannitol (4.0%). Each of the fibrinogen solution (2.5 mL) was soaked evenly into a bioabsorbable synthetic non-woven fabric made of polyglycolic acid (Neoveil, Gunze Limited, 5×10 cm, thickness 0.15 mm). This sample, after being frozen and lyophilized for 24 hours, was used as a sample of a supporting material holding fibrinogen (fibrinogen held at 4 mg/cm²).

### Example 1

### Test Estimating Effect to Inhibit Scar Contracture in Guinea Pig Deficient Skin Model

### (1) Method of Experiment

Induction of anesthesia was performed to guinea pig (male, Hartley, 5 weeks old, Japan SLC, Inc.) with ketamine (DAITCHI SANKYO COMPANY, LIMITED), diazepam (Takeda Pharmaceutical Company Limited) and anesthesia continued by inhalation of isoflurane (Merck) through a mask. At the skin of the back, several full-thickness skin wounds of 1.5 cm square were generated and treated as indicated in each of Groups hereinbelow. For groups 1 and 3, time required for application was measured. On Day 5, Day 8, Day 10, Week 2 and Week 3 after the treatment, photographs of the wound sites were taken and an area of the wounds was obtained by image analysis. Also, on Day 5, Day 10, Week 2, Week 3 and Week 6 after the treatment, each four animals were sacrificed by euthanasia and the treated sites were sampled for histopathology.

### (2) Groups

Group 1: Wound-covering material (the present invention): After a fibrinogen solution (Bolheal (registered trade mark), Juridical Foundation The Chemo-Sero-Therapeutic Research Institute) was rubbed into the wound sites, a bioabsorbable synthetic non-woven fabric (Neoveil (registered trade mark), Gunze Limited) was applied thereto, onto which the fibrinogen solution and a thrombin solution (Bolheal (registered trade mark), Juridical Foundation The Chemo-Sero-Therapeutic Research Institute) were sprayed.
Group 2: No treatment (negative control): No treatment was carried out to let it be an open wound.
Group 3: Terudermis: Terudermis (registered trade mark; Terumo), a graft for deficiency of the dermis, was applied to the wound sites and sutured with Nylon thread by single-knot suture.

### (3) Results

### (i) Time for Application

Time necessary for application was 368.0±27.2 seconds (mean: n=8) for Terudermis whereas it was 44.2±6.1 seconds (mean: n=9) for the wound-covering material. Since the wound-covering material had adhesiveness of its own with no necessity of suture, it could be applied in shorter time, about 1/9, than Terudermis.

### (ii) Area of Wound Surface

A retention rate of an area of wound was 95.5±5.75% (n=6) for the wound-covering material whereas it was 73.6±16.21% (n=5) for no treatment and 73.1±18.02% (n=5) for Terudermis. As such, contraction of wound was inhibited for the wound-covering material as compared to no treatment and Terudermis, on Day 8 after treatment.

(iii) Histopathological Examination

### <Comparison Between Groups>

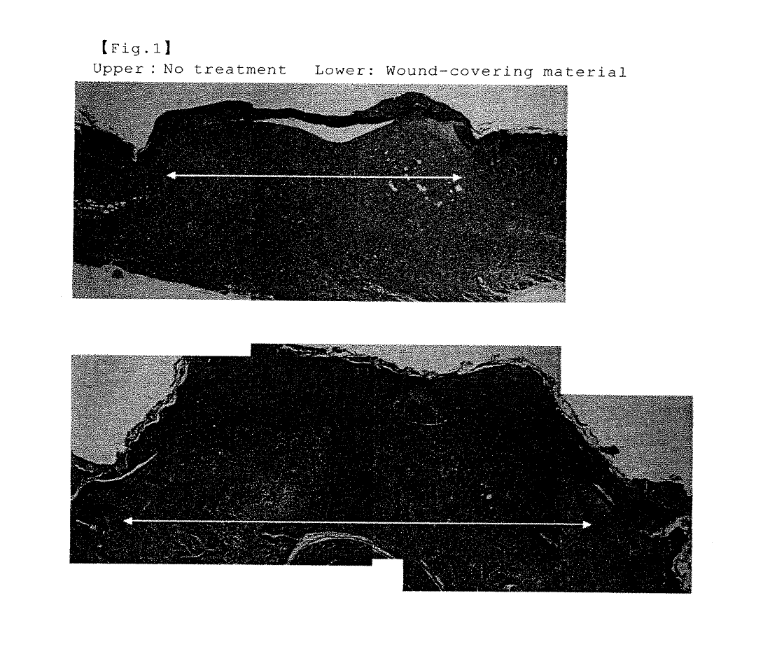

For the wound-covering material, edema and retention of blood and exudates under the crust on Day 5 were lighter than those of no treatment and Terudermis. On Day 10, a depressed area after wound was filled with granulation tissue for every Groups but a distance between the original collagen layers was the most kept for the wound-covering material as compared to no treatment and Terudermis (FIG. 1).

The difference observed on Day 5 may be attributed to the sealing effect of the wound-covering material of the present invention which inhibited retention of blood and exudates. Since retained substance may cause pain or retarding in repair, decrease in these would be expected.

The difference observed on Day 10 may be attributed to appropriate strength of the wound-covering material of the present invention after application which ensures space where granulation tissue is formed and inhibits wound contraction to thereby keep a distance between the original collagen layers. Thus, decrease in scar contracture and pain would be expected by the use of the wound-covering material.

<Cure by Wound-Covering Material with Passage of Time>
Day 5: Under the wound-covering material was there a thin layer of exudates, under which fibrosis and regeneration of the epithelium (in portion) from the peripheral region was observed.
Day 10: The wound-covering material was peeled off. The epithelium was regenerated from the peripheral region and under the epithelium neovascularization and formation of granulation tissue were observed.
Week 2: Regeneration of the epithelium was prominent and in some cases complete coverage was observed.
Week 3: Complete coverage by the epithelium was observed. Collagen was seen in the granulation tissue and repair of the dermis and subcutaneous tissue progressed.
Week 6: Continuity of collagen in the dermis was almost recovered. Also, the skin appendage was almost repaired.

As such, no excessive inflammation etc. was observed in the passage of cure with the wound-covering material of the invention and the layers of the epithelium and the dermis were promptly reconstructed.

### Example 2

### Observation of Histopathology During Passage of Replacement with Auto-Tissue Under Moisture Condition: Guinea Pig Deficient Skin Model

### (1) Method of Experiment

Introductory anesthesia was performed to guinea pig (male, Hartley, 5 weeks old, Japan SLC, Inc.) with ketamine (DAIICHI SANKYO COMPANY, LIMITED), diazepam (Takeda Pharmaceutical Company Limited) and anesthesia continued by inhalation of isoflurane (Merck) through a mask. At the skin of the back, several full-thickness skin wounds of 1.5 cm square were generated and treated as indicated in each of Groups hereinbelow. On Day 8, Week 2 and Week 3 after the treatment, the animals were sacrificed by euthanasia and the treated sites were sampled for histopathology.

### (2) Group

Group 1: Wound-covering material: After a fibrinogen solution (Bolheal (registered trade mark), Juridical Foundation The Chemo-Sero-Therapeutic Research Institute) was rubbed into the wound sites, a bioabsorbable synthetic non-woven fabric (Neoveil (registered trade mark), Gunze Limited) was applied thereto, onto which the fibrinogen solution and a thrombin solution (Bolheal (registered trade mark), Juridical Foundation The Chemo-Sero-Therapeutic Research Institute) were sprayed, which was further covered with a covering material for retaining moisture.

### (3) Histopathological Examination

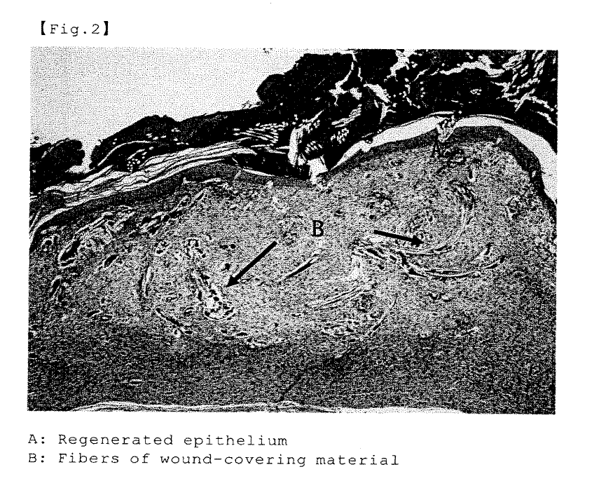

Day 8: A depressed area after wound was filled with granulation tissue and regeneration of the epithelium from the peripheral region was observed.
Week 2: Regeneration of the epithelium was prominent and in some cases complete coverage was observed.
Week 3: With the bioabsorbable non-woven fabric of the wound-covering material and fibrin clot serving as a scaffold, hyperplasia of the connective tissue and neovascularization were observed and the wound-covering material was being replaced with auto-tissue (FIG. 2). It was revealed that, under moisture condition, the wound-covering material was not naturally peeled off after cure of the wound but was replaced with auto-tissue.

### INDUSTRIAL APPLICABILITY

The wound-covering material of the present invention, via its own adhesiveness, may easily be stuck to a deficient area of the skin to protect the wound surface. Besides, the wound-covering material of the present invention, as alleviating scar contracture and pain, may reconstruct deficient tissue of the skin in a prompt and good manner through its biocompatibility and positive repair of tissue. Therefore, the wound-covering material of the present invention may be applied as a wound-covering material or an artificial skin for a deficient area of the skin caused by trauma, surgical operation or burn.

1. A wound-covering material, comprising:
a first layer comprising thrombin, fibrinogen, and a bioabsorbable supporting material; and
a second layer comprising a covering material;
wherein the bioabsorbable supporting material is a substrate and the first layer is overlaid with the second layer.

2. The wound-covering material of claim 1, wherein:
(1) the bioabsorbable supporting material comprises the thrombin, and the fibrinogen;
(2) a first bioabsorbable supporting material comprises the thrombin, and a second bioabsorbable supporting material comprises the fibrinogen; or
(3) the bioabsorbable supporting material comprises no thrombin and no fibrinogen, and
wherein the thrombin and the fibrinogen are separated from each other.

3. The wound-covering material of claim 1, wherein the bioabsorbable supporting material is a polymer comprising, in reacted form, polyglycolic acid or polylactic acid or a copolymer comprising, in reacted form, glycolic acid and lactic acid.

4. The wound-covering material of claim 1, wherein the bioabsorbable supporting material comprises a non-woven fabric.

5. The wound-covering material of claim 1, wherein the bioabsorbable supporting material comprises a non-woven fabric comprising, in reacted form, polyglycolic acid.

6. The wound-covering material of claim 1, further comprising at least one additive selected from the group consisting of Blood Coagulation Factor XIII, a protease inhibitor, calcium chloride, albumin, sodium chloride, sodium citrate, and a non-ionic detergent.

7. (canceled)

8. The wound-covering material of claim 1, wherein the covering material comprises silicon or a polyurethane.

9. The wound-covering material of claim 1, wherein the wound-covering material is suitable for repairing a deficient area of skin.

10. A wound-covering material kit, comprising:
a bioabsorbable supporting material comprising thrombin;
a container comprising fibrinogen; and
a covering material.

11. The kit of claim 10, wherein the bioabsorbable supporting material is a polymer comprising, in reacted form, polyglycolic acid or polylactic acid or a copolymer comprising, in reacted form, glycolic acid and lactic acid.

12. The kit of claim 10, wherein the bioabsorbable supporting material comprises a non-woven fabric.

13. The kit of claim 10, wherein the bioabsorbable supporting material comprises a non-woven fabric comprising, in reacted form, polyglycolic acid.

14. The kit of claim 10, further comprising at least one additive selected from the group consisting of Blood Coagulation Factor XIII, a protease inhibitor, calcium chloride, albumin, sodium chloride, sodium citrate, and a non-ionic detergent.

15. The kit of claim 14, wherein the bioabsorbable supporting material comprises calcium chloride.

16. The kit of claim 14, wherein the container comprises Blood Coagulation Factor XIII.

17. The kit of claim 10, wherein the bioabsorbable supporting material is obtained by a process comprising immersing a bioabsorbable supporting material in a solution comprising thrombin, and then lyophilizing the bioabsorbable supporting material.

18. (canceled)

19. The kit of claim 10, wherein the covering material comprises silicon or a polyurethane.

20. The kit of claim 10, wherein the kit is suitable for repairing a deficient area of skin.

21. A wound-covering material kit, comprising:
a first bioabsorbable supporting material comprising thrombin;
a second bioabsorbable supporting material comprising fibrinogen; and
a covering material.

22. The kit of claim 21, wherein the first and second bioabsorbable supporting materials are a polymer comprising, in reacted form, polyglycolic acid or polylactic acid or a copolymer comprising, in reacted form, glycolic acid and lactic acid.

23. The kit of claim 21, wherein the first and second bioabsorbable supporting materials comprise a non-woven fabric.

24. The kit of claim 21, wherein the first and second bioabsorbable supporting materials comprise a non-woven fabric comprising, in reacted form, polyglycolic acid.

25. The kit of claim 21, further comprising at least one additive selected from the group consisting of Blood Coagulation Factor XIII, a protease inhibitor, calcium chloride, albumin, sodium chloride, sodium citrate, and a non-ionic detergent.

26. The kit of claim 25, wherein the first bioabsorbable supporting material comprises calcium chloride.

27. The kit of claim 25, wherein the second bioabsorbable supporting material comprises a non-ionic detergent.

28. The kit of claim 21, wherein the first and second bioabsorbable supporting material are obtained by a process comprising immersing a bioabsorbable supporting material in a solution comprising thrombin or fibrinogen, respectively, and then lyophilizing the bioadsorbable supporting material.

29. (canceled)

30. The kit of claim 21, wherein the covering material comprises silicon or a polyurethane.

31. The kit of claim 21, wherein the kit is suitable for repairing a deficient area of skin.

32. A wound-covering material kit, comprising:
a first container comprising thrombin;
a second container comprising fibrinogen;
a bioabsorbable supporting material; and
a covering material.

33. The kit of claim 32, wherein the bioabsorbable supporting material is a polymer comprising, in reacted form, polyglycolic acid or polylactic acid or a copolymer comprising, in reacted form, glycolic acid and lactic acid.

34. The kit of claim 32, wherein the bioabsorbable supporting material comprises a non-woven fabric.

35. The kit of claim 32, wherein the bioabsorbable supporting material comprises a non-woven fabric comprising, in reacted form, polyglycolic acid.

36. The kit of claim 32, further comprising at least one additive selected from Blood Coagulation Factor XIII, a protease inhibitor, calcium chloride, albumin, sodium chloride, sodium citrate, and a non-ionic detergent.

37. The kit of claim 36, wherein the first container comprises calcium chloride.

38. The kit of claim 36, wherein the second container comprises Blood Coagulation Factor XIII.

39. (canceled)

40. The kit of claim 32, wherein the covering material comprises silicon or a polyurethane.

41. The kit of claim 32, wherein the kit is suitable for repairing a deficient area of skin.
